(19) United States
(12) Patent Application Publication
(10) Pub. No.: US 2010/0057000 A1
(43) Pub. Date: Mar. 4, 2010

(54) MALECOT WITH TEXTILE COVER

(75) Inventors: Jeffry S. Melsheimer, Springville, IN (US); Shyam Kuppurathanam, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/199,586

(22) Filed: Aug. 27, 2008

(51) Int. Cl. A61M 25/04; D03D 15/00

(52) U.S. Cl. 604/103.03; 139/420 A

(57) ABSTRACT

A malecot device includes a textile material covering its deployable malecot wing portion. The textile material is woven in a manner allowing it to be compactly disposed around an undeployed malecot and then expanded to cover malecot wings when those wings are deployed to an expanded outer device diameter.

### TECHNICAL FIELD

The present patent application relates generally to medical devices, particularly to a malecot device configured with an expandable member and a textile cover around the expandable member.

### BACKGROUND

Malecot devices are well-known within the medical art, including as expandable retention members for gastronomy tubes, jejunal feeding tubes, drainage catheters and other applications. In particular, they are known for use in catheter retention applications and other applications where it is desirable to have an expandable member to hold a tubular device within a patient body. For example, malecots are known for use in securing drainage tubes and feeding tubes. A proximal portion of the deployable wing portions of many current malecot designs may, when deployed, include edges that can create irritating friction or otherwise potentially undesirable tissue contact, particularly when in contact with tissue over any length of time. Therefore, it may be advantageous to provide a malecot device with an improved atraumatic or minimally traumatic expandable region including a deployable wing portion.

### BRIEF SUMMARY

In one aspect, embodiments of the present invention may include a malecot device that includes an elongate generally tubular body with an expandable region. The expandable region includes a proximal end and a distal end with an elongate body lumen disposed through its length. In one such embodiment, the body may include a plurality of generally parallel elongate slits defining lateral sides to form a plurality of foldable malecot wings configured to be deployed by folding outward of a middle portion thereof. A woven textile material substantially circumferentially surrounds the expandable body region, where the woven textile material includes warp and weft fibers oriented at about 45 degrees relative to a longitudinal axis of the elongate tubular body and is configured to form a substantially continuous expandable covering that will accommodate the malecot wings when they are in a deployed configuration.

In another aspect, embodiments of the present invention may include a malecot device that includes an elongate generally tubular body with a proximal end, a distal end, and an elongate lumen disposed through its length. In such embodiments, the body may include an expandable region that includes a plurality of generally parallel elongate slits defining lateral sides of a plurality of malecot wings. The malecot wings are configured to deploy by folding outward from a central longitudinal axis of the body upon movement of the proximal and distal ends longitudinally toward each other. The device further includes a textile material substantially circumferentially surrounding the expandable body region.

In still another aspect, embodiments of the present invention may include a method of making a malecot device, where the method includes the steps of: providing a malecot body including an expandable region that includes a plurality of generally parallel elongate slits defining lateral sides of a plurality of malecot wings; providing a woven textile material configured as a tube; and disposing at least one end of the woven textile material around the malecot body such that a portion of the material substantially covers the expandable region.

### BRIEF DESCRIPTION OF THE DRAWINGS

FIGS. 1A and 1B show, an uncoated malecot device in, respectively, an undeployed and a deployed configuration;

FIG. 2A shows an undeployed textile-covered expandable region of a malecot device embodiment;

FIG. 2B shows the device of FIG. 2A, including the underlying malecot structure;

FIG. 2C shows the device of FIGS. 2A-2B with the malecot's expandable region in a deployed configuration;

FIG. 3A shows a pleated textile covering an undeployed expandable region of an malecot device embodiment;

FIG. 3B shows the device of FIG. 3A with the malecot's expandable region in a deployed configuration;

FIG. 3C shows the device of FIGS. 3A-3B including the underlying malecot structure;

FIG. 3D shows an end-perspective, transverse section view of the device of FIGS. 3A-3C;

FIGS. 4 and 4A show, respectively top and transverse section views of a dual-layer/tubular textile material as it may be formed on a loom;

FIG. 5A shows how a user may cut a tubular unit of a textile material;

FIG. 5B shows how a user may finish portions of the tubular unit of FIG. 5A;

FIGS. 6A and 6B depict the elongation and foreshortening capabilities of a textile material tubular unit;

FIGS. 7A and 7B depict the radial expandability and elongation capabilities of a textile material tubular unit when mounted onto a malecot device; and

FIG. 8 shows another configuration of a textile material as it may be formed on a loom.

### DETAILED DESCRIPTION OF THE DRAWINGS AND VARIOUS EMBODIMENTS

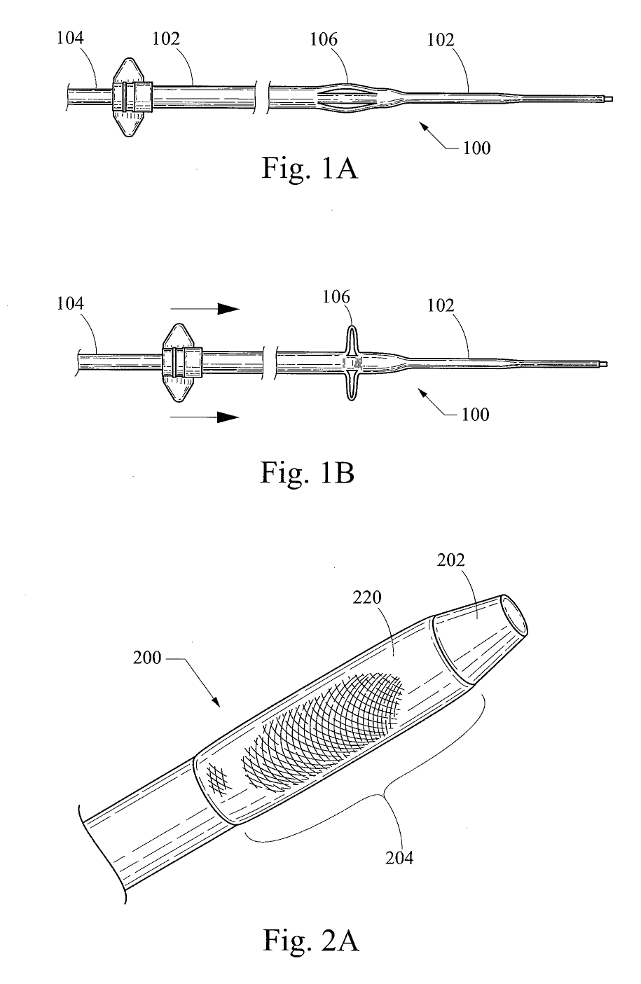

As shown in FIG. 1A, an uncoated malecot design 100 may include an outer sheath 102 having a distal portion that is affixed to an introducer device 104. The malecot 100 may be introduced with the outer sheath 102 in an undeployed, near-cylindrical configuration. As shown in FIG. 1B, the expandable malecot wing portion 106 of the outer sheath 102 may then be deployed by moving a proximal portion of the outer sheath 102 distally relative to the introducer device 104 and/or by moving the introducer device 104 proximally relative to the outer sheath 102. The introducer device 104 generally must be held in place in order to keep the malecot wing portion 106 deployed.

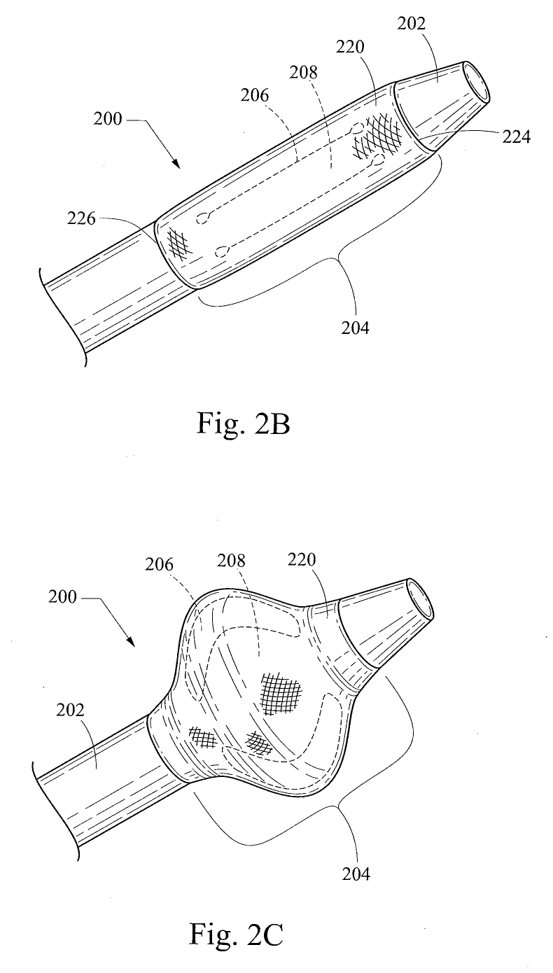

One embodiment of a textile-covered malecot device 200 is described with reference to FIGS. 2A-2C. FIG. 2A illustrates a perspective view of the distal end portion of the malecot device 200, which is illustrated as a gastronomy tube device. The device 200 includes an elongate generally tubular body 202 with an expandable malecot region 204. The expandable malecot region 204 is substantially circumferentially surrounded by a textile material 220 that forms a malecot cover, which - in its unexpanded configuration - generally includes the geometry of a right cylinder.

As shown in FIG. 2B, the expandable malecot region 204 of the body 202 includes generally parallel slits 206 that define lateral margins of malecot wings 208. The material 220 is attached to the body 202 at least around a portion of its distal circumference 224. Preferably, the material 220 is attached to the body 202 around its distal circumference 224 and around its proximal circumference 226. FIG. 2C illustrates that the expandable malecot region 204 is deployed by folding a central region of the malecot wings 208 outward relative to the central longitudinal axis of the body 202 such that a proximal portion and a distal portion of each malecot wing 208 is folded together toward each other. As the proximal and distal wing portions are folded toward each other, an intermediate portion is bent and translated outward relative to a central longitudinal axis of the body 202. When this is done, the material 220 expands to accommodate the deployed malecot wings 208 and form a generally enclosed surface around a volume substantially defined by the malecot wings 208. In the illustrated embodiment, the deployed malecot device 200 provides a geometry that is generally that of a right cylinder with a convex intermediate portion having a greater diameter than its end portions (i.e., it is bowed out in the middle, and—in the present example—provides a generally circular transverse cross-sectional profile). Preferably, the material 220 forms a tautly-pulled surface over the exterior surfaces of the malecot wings 208 and across the expanded spaces of the malecot slits 206.

As will be apparent to those of skill in the art, when an uncovered malecot design 100 of the type shown in FIGS. 1A-1B is placed into contact with a body surface, the contact forces will be concentrated along the faces of the malecot wings 106. The edges of those malecot wing faces may irritate tissue where contact occurs. In contrast, a covered malecot device of the present invention such as, for example, the device 200 illustrated in FIGS. 2A-2C will provide a generally smooth surface without edges and distribute the contact forces generally uniformly over a greater surface area such that they are more diffuse and have a reduced likelihood of causing irritation or injury.

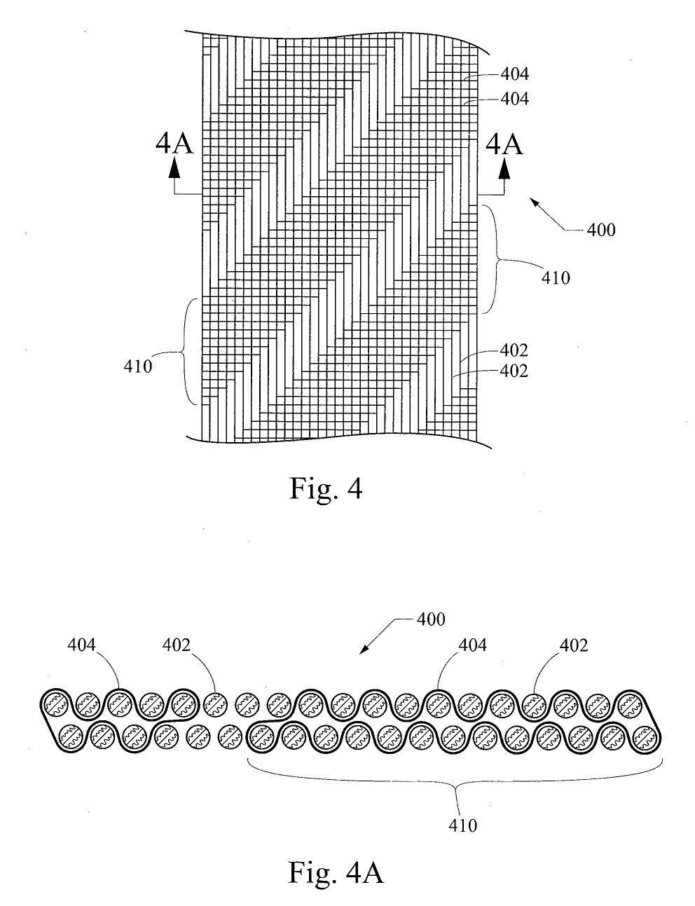

The generally tubular body may be formed in the same manner as known malecot devices using biocompatible materials known to those of skill in the art. The textile material of the malecot cover preferably will provide a substantially smooth exterior surface. Exemplary textile materials and construction are described below with reference to FIGS. 4-8. Other materials that may be suitable, or that may be adapted to be suitable, for use with embodiments of the present invention include materials known and used in stent-grafts, fabric stent coverings, percutaneous transluminal angioplasty balloons or other balloons used with endoscopic procedures. A malecot cover used within the scope of the present invention may be configured as a “pseudo-balloon” using a balloon design such as, for example, those disclosed in U.S. Pat. No. 4,637,396; 6,592,550; or U.S. Pat. App. No. 2006/0135983, each of which is incorporated by reference herein in its entirety, as well as other balloon devices and varieties thereof that are known in the art, and particularly those that provide a textile material that may be introduced in a low profile around a tubular device and then expanded to present a generally smooth outer biocompatible outer surface.

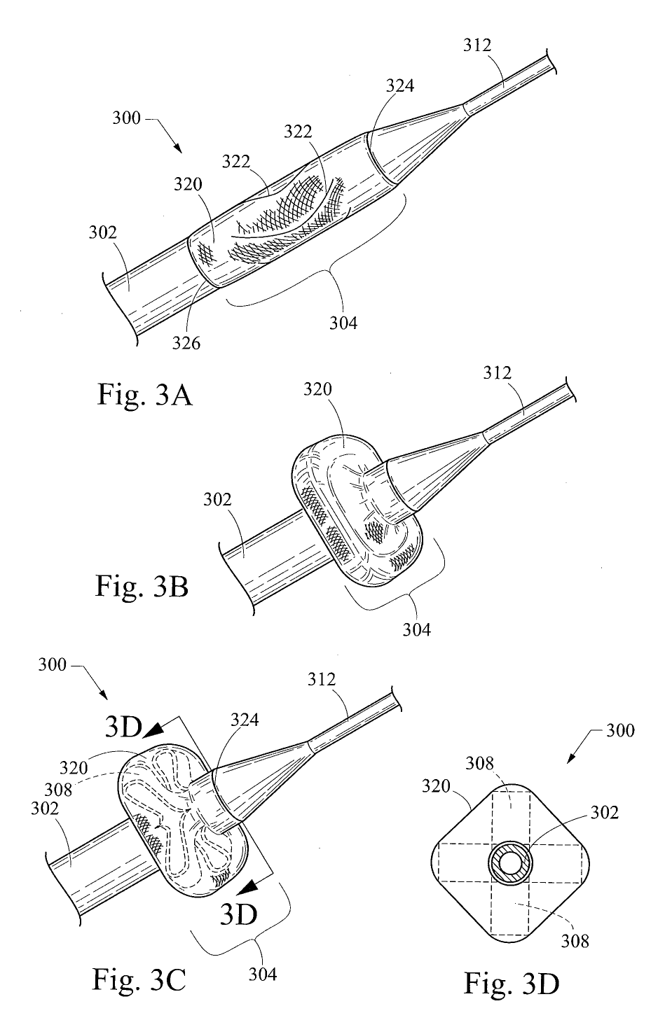

Another embodiment of a textile-covered malecot device is described with reference to FIGS. 3A-3D. FIG. 3A illustrates a perspective view of the distal end portion of an unexpanded malecot device, which is illustrated as a jejunostomy tube device 300. The device 300 includes an elongate generally tubular body 302 having a distal end region 312 that is longer than the distal portion of the gastronomy tube device 200. The body portion 302 of the device 300 includes an expandable malecot region 304. The expandable malecot region 304 is substantially circumferentially surrounded by a textile material 320 that forms a malecot cover. In the embodiment illustrated, the malecot cover 320 generally includes the geometry of a right cylinder when in its unexpanded configuration, and it may be tightly pleated to provide a smooth outer profile. The textile material 320 is shown as including pleats 322, which may be incorporated when the unexpanded intermediate portion of the material 320 has a larger circumference than the undeployed outer circumference of the body 302. In some embodiments, the material 320 may be sufficiently flexible that it can accommodate malecot expansion without needing to have a pleated configuration while disposed around the undeployed expandable malecot region.

The expandable malecot region 304 of the body 302 includes slits that define lateral margins of malecot wings 308. The material 320 is attached to the body 302 at least around a portion of its distal circumference 324. Preferably, the material 320 is attached to the body 302 around its distal circumference 324 and around its proximal circumference 326 (i.e. at locations adjacent to each end of the malecot region 304). FIGS. 3B-3D illustrate that the expandable malecot region 304 is deployed by folding the malecot wings 308 outward relative to the central longitudinal axis of the body 302. When this is done, the material 320 expands to accommodate the deployed malecot wings 308 and form a generally enclosed surface around a volume substantially defined by the malecot wings 308. In the illustrated embodiment, the deployed malecot device 300 provides a geometry that is generally that of a right cylinder with a convex intermediate portion having a greater diameter than its end portions (i.e., it is bowed out in the middle, and in the present example, as shown in FIG. 3D, which is a partial section view taken along line 3D-3D of FIG. 3C., provides a generally rounded square or an irregular octagonal cross-sectional profile). Preferably, the material 320 forms a tautly-pulled surface over the exterior surfaces of the malecot wings 308 and across the expanded spaces of the malecot slits.

A textile material 400 and method of making and using the same with a malecot device are described with reference to FIGS. 4-7B. FIG. 4 shows an example of how the textile material 400 may be formed on a loom. The fibers are shown as being spaced apart for the sake of illustrative clarity, and the figures are not drawn to scale for the same reason, but it should be appreciated that preferred textile materials will include a fairly tight woven structure with warp and weft fibers that may have similar or dissimilar cross-sectional profiles (e.g., circular or non-circular) and areas that will provide a substantially smooth fabric such as, for example, the textile material used in angioplasty balloons and other medical-use balloons, although materials suitable for use in the present invention need not be able to retain a fluid. Exemplary fiber materials that may be used to form a material 400 include nylon, polyester, PTFE, and other polymer fibers, although it is also contemplated that metallic alloy fibers may also be used within the scope of the present invention.

FIG. 4 shows a weaving pattern of a material 400 with vertical warp fibers 402 and horizontal weft fibers 404. The weft fibers 404 are oriented at about 90 degrees relative to the warp fibers 402. FIG. 4A shows a transverse cross-section view taken along line 4A-4A of FIG. 4. The loom preferably is configured for weaving a continuous sheet of material formed as a tube. Specifically, with reference to FIG. 4A, the weft fibers 404 are woven through an upper layer 402a of warp fibers 402 and then continuously woven through a lower layer 402b (not visible in FIG. 4) of warp fibers 402, and back again to form a flattened tube 410. As shown in FIG. 4, separate tubular units 410 may be formed in a series. In the illustrated embodiment, the tubular unit 410 is woven in a flattened configuration, but those of skill in the art will appreciate that a material forming a tubular unit as described herein may be formed about a mandrel or in some other weaving configuration.

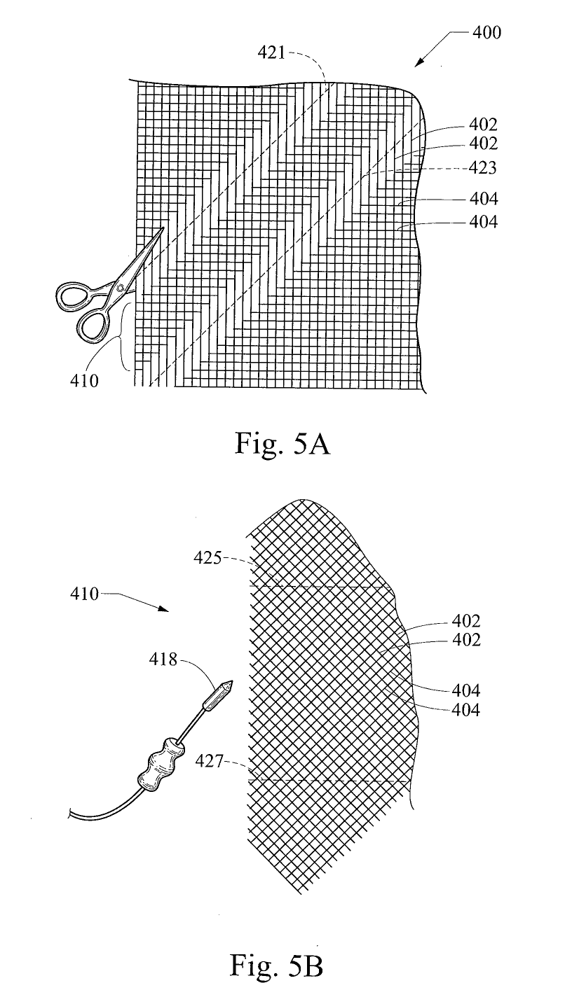

As is also shown in FIG. 4, the longitudinal axis of each tubular unit 410 is formed at about a 45 degree angle relative to the warp fibers 402. FIG. 5A illustrates that a user may cut through the unwoven portion of warp fibers 402 (including both upper and lower layers 402a, 402b) at about a 45 degree angle along lines 421 and 423, parallel with the woven edges of the tubular unit 410. Next, as shown in FIG. 5B, a user may cut the ends of the tubular unit 410 along lines 425, 427 such that it generally has a geometry of a right cylinder when formed to have a generally circular transverse cross-section. After these cuts have served to trim the tubular unit 410, a user may finish the tubular unit 410 by using a selvage means. Selvage means may include heat sealing the cut ends of the warp and/or weft fibers back onto themselves or onto adjacent fiber, including that, along the sides parallel to the longitudinal axis in FIG. 5B, the cut ends of warp fibers from the upper layer may be heat-sealed with a soldering iron 418 or other heating means (e.g. laser, welding means). In some embodiments, the cut ends of the warp and/or weft fibers may be connected (e.g., by heat-sealing or adhesive means) to adjacent cut ends of warp and/or weft fibers to form one or more substantially continuous helices that are oriented at about 90 degrees relative to the helices formed by the weft fibers. Other selvage means may include stitching in a separate fiber to secure cut fibers, using an adhesive to seal cut ends of fibers, rolling or tucking the cut ends of fibers relative to other fibers, using any other selvage means known or developed in the art, or any combination thereof.

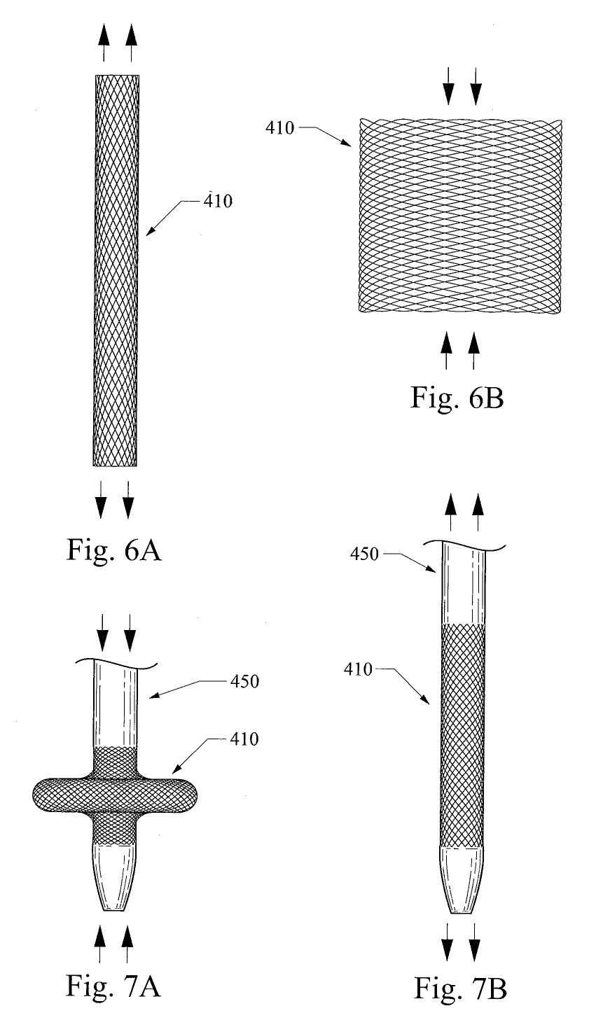

FIG. 6A shows that the tubular unit 410 illustrated in FIG. 5B can be pulled lengthwise in a manner that flexes the weave and elongates the tube 410. FIG. 6B shows that the tube 410 can be longitudinally compressed/foreshortened in a manner that longitudinally compresses the weave and demonstrates its radial expandability. As depicted in FIGS. 7A and 7B, both of these properties may be exploited by mounting at least one end, and preferably both ends, of the woven tube 410 onto a malecot device 450. FIG. 7A shows the tube 410 mounted onto the malecot device 450, with an expandable region of the device 450 being deployed (similar to that shown in FIG. 2C, but with greater longitudinal compression). The radial expandability of the tube 410 allows its intermediate portion to flex radially outward and conform to the expanded malecot in the manner shown in FIG. 7A. As illustrated in FIG. 7B, upon re-elongation/undeployment of the expandable region of the malecot device 450, the tube 410 will resume its generally right-cylindrical geometry.

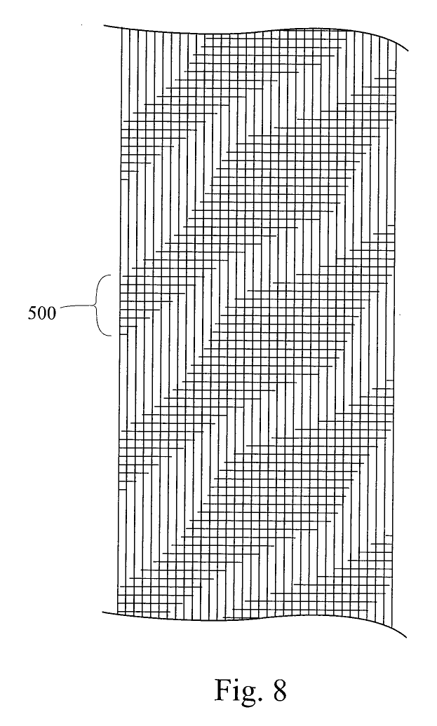

Another embodiment of a textile material tube 500 of the present invention is illustrated with reference to FIG. 8. The tube 500 may be formed in substantially the same manner as that described above with reference to FIGS. 5-5B. However, a convex intermediate portion is incorporated during the weaving process. Those of skill in the art will appreciate that this configuration of a tube 500 will provide a generally right-cylindrical tube geometry with an intermediate outer surface region that is convex in a manner forming a generally symmetrical bulge around the outer circumference of the tube 500. A tube 500 with this increased intermediate circumference may be pleated when mounted onto a malecot device such that, when mounted, it would have an appearance similar to the malecot cover 320 illustrated in FIG. 3A. Those of skill in the art will appreciate that the textile materials described with reference to FIGS. 4-8 may have mechanical properties similar to braided material in spite of being woven rather than braided. One may use a braided material rather than a woven material, but such would not be considered as equivalent to the preferred woven material.

Many modifications and other aspects of the invention may come to mind to one skilled in the art to which this invention pertains upon having the benefit of the teachings presented in the foregoing description; and it will be apparent to those skilled in the art that variations and modifications of the present invention can be made without departing from the scope or spirit of the invention. Although specific terms are employed herein, they are used in a generic and descriptive sense only and not for purposes of limitation. Therefore, it is to be understood that the invention is not to be limited to the specific aspects disclosed and that modifications and other aspects are intended to be included within the scope of the claims, which define the present invention.

1. A malecot device comprising:
an elongate tubular body with an expandable region that includes a plurality of foldable malecot wings configured to be deployed by folding together a proximal and a distal portion thereof;
a woven textile material substantially circumferentially surrounding the expandable body region, the woven textile material comprising warp and weft fibers oriented at about 45 degrees relative to a longitudinal axis of the elongate tubular body and configured to form a substantially continuous expandable covering that will accommodate the malecot wings when they are in a deployed configuration.

2. The malecot device of claim 1, where the textile material comprises a woven material generally formed as a tube and having its warp and its weft disposed at about 45 degrees relative to each other.

3. The malecot device of claim 1, where the textile material substantially circumferentially surrounding the expandable body region is configured to allow radial expansion of an outer diameter of the expandable region during a deployment of the malecot wings.

4. The malecot device of claim 3, where, upon deployment of the malecot wings, the textile material is configured to expand and form a generally enclosed surface around a volume substantially defined by the malecot wings.

5. The malecot device of claim 4, where the generally enclosed surface provides a generally atraumatic surface.

6. A malecot device comprising:
an elongate generally tubular body including a proximal end and a distal end with an elongate lumen disposed through its length;
where the body comprises an expandable region that includes a plurality of malecot wings;
where the malecot wings are configured to deploy by folding and translating an intermediate portion outward from a central longitudinal axis of the body upon moving the proximal and distal ends longitudinally nearer to each other; and
further comprising a textile material substantially circumferentially surrounding the expandable body region.

7. The malecot device of claim 6, where the textile material comprises a selected one of a braided material or a woven material.

8. The malecot device of claim 6, where the textile material comprises a woven material, the woven material comprising at least one of a warp or a weft is disposed at an angle of less than 90 degrees relative to the central longitudinal body axis.

9. The malecot device of claim 8, where the at least one of the warp or the weft is disposed at an angle of about 45 degrees relative to the central longitudinal body axis.

10. The malecot device of claim 6, where the textile material comprises a woven material having a warp and a weft disposed at about 90 degrees relative to each other.

11. The malecot device of claim 6, where the textile material comprises a polymer selected from polyester, nylon, PTFE, and any combination thereof.

12. The malecot device of claim 6, where the textile material comprises a general tubular shape.

13. The malecot device of claim 12, where the textile material comprises a selected one of a right cylinder or a cylinder having a convex intermediate portion of greater diameter than its end portions.

14. The malecot device of claim 12, where the textile material comprises a generally cylindrical shape having a convex intermediate portion of greater diameter than its end portions, and where the convex intermediate portion is generally pleated when the malecot is in an unexpanded configuration, in a manner providing an outer diameter that is substantially the same as adjacent end portions.

15. A method of making a malecot device, the method comprising the steps of:
providing a malecot body including an expandable region that includes a plurality of foldable malecot wings;
providing a woven textile material configured as a tube; and
disposing the woven textile material around the malecot body such that a portion of the material substantially covers the expandable region; and
attaching at least one end of the woven textile material to the malecot body adjacent the malecot wings.

16. The method of claim 15, where the step of providing a woven textile material further comprises the step of weaving polymer fibers such that weft fibers are oriented at about 90 degrees relative to warp fibers, and the warp and weft fibers are both oriented at about 45 degrees relative to a longitudinal axis of the tube.

17. The method of claim 16, where the step of weaving polymer fibers further comprises weaving the fibers on a loom in a dual layer manner to provide the tube, and trimming the fibers such that the tube generally forms a right cylindrical tube.

18. The method of claim 16, where the step of weaving polymer fibers further comprises treating trimmed portions of the tube by a selvage means.

19. The method of claim 18, where the selvage means is selected from a group consisting of stitching, heat treating, adhesive sealing, rolling, and any combination thereof.

20. The method of claim 15, where the woven textile material configured as a tube comprises first and second ends, and the step of attaching further comprises attaching the first end to the malecot body adjacent a proximal end of the expandable region and attaching the second end to the malecot body adjacent a distal end of the expandable region.
